(19) United States
(12) Patent Application Publication
(10) Pub. No.: US 2013/0137602 A1
(43) Pub. Date: May 30, 2013

(54) ARBITRARY ASSEMBLY OF NANO-OBJECTS INTO DESIGNED 1D AND 2D ARRAYS

(71) Applicants: Oleg Gang, Setauket, NY (US); Daniel van der Lelie, Chapel Hill, NC (US)

(72) Inventors: Oleg Gang, Setauket, NY (US); Daniel van der Lelie, Chapel Hill, NC (US)

(73) Assignee: Brookhaven Science Associates, LLC, Upton, NY (US)

(21) Appl. No.: 13/583,517

(22) PCT Filed: Mar. 7, 2011
(86) PCT No.: PCT/US11/27393

§ 371 Date: Jan. 7, 2013

Related U.S. Application Data
(60) Provisional application No. 61/311,991, filed on Mar. 9, 2010.

(51) Int. Cl. C12Q 1/68

(52) U.S. Cl. CPC C12Q 1/6837
USPC 506/16; 506/30

(57) ABSTRACT

The present invention is directed to nanoscale fabrication of nano-materials with application in electronics, energy conversion, bio-sensing and others. Specifically, the invention is directed to arbitrary, that is periodic and non-periodic, assembly of nano-objects on I D and 2D arrays. The present invention utilizes self-organization properties of nanoscale bio-encoded building blocks, programmability of biomolecular interactions, and simple processing techniques for providing arbitrary by-design fabrication capability. Specifically, the present invention utilizes double stranded DNA attached to a surface and intercalating PNA-DNA hybrids attached to nano-objects to bind the nano-objects to the dsDNA in a site specific manner. The present invention allows for an integration of a large number of nano-components in unified well-defined systems. Accordingly, the present invention is applicable for fabrication of I D and 2D structures of various by-design placements of nano-objects of multiple types, including metal, semiconducting and organic nano-objects.

This application is an International PCT application, which claims the benefit of U.S. Provisional Application No. 61/311,991, filed on Mar. 9, 2010 which is hereby incorporated by reference in its entirety.

The present invention was made with government support under Contract No. DE-AC02-98CH 10886 awarded by the U.S. Department of Energy. The United States government has certain rights in the invention.

### BACKGROUND

I. Field of the Invention

The invention is directed to nanoscale fabrication which can be used for the fabrication of broad classes of nano-materials with application in electronics, energy conversion, bio-sensing and others. Specifically, the invention is directed to arbitrary assembly of nano-objects on arrays.

II. Background of the Related Art

The nanoscience revolution has led to the rapid development of a diversity of remarkable nanoscale objects including metallic and semiconductor nanoparticles, carbon based nanomaterials and supramolecular organic complexes. In order to construct complex functional systems from these nanoscale objects, new methods of material assembly are required. While conventional lithographic methods have been proven to provide robust and versatile fabrication approaches, their limited resolution, increasing cost of fabrication of small features on large areas, serial nature of fabrication process, and limited ability to integrate newly developed synthetic nanoscale functional blocks call for new methods in material and device fabrication. Conventional self-assembly is promising for the creation of large scale structures since it relies on the intrinsic ability of the system's components to self-organize in particular structures based on their mutual interactions and entropic effects. Conventional self-assembly can be assisted with external fields, stimuli, patterns, and the like.

Although the conventional approach offers an ease of fabrication, it often cannot compete with lithographic methods for a number of reasons. First, there is rarely a rational design of final structures because of the complex relationship between component interactions of a system and the final structure. Second, self-assembly is mostly limited to assembly of similar components or only few types of different components, which is a serious drawback for fabrication of complex structures. Third, and more importantly, structures fabricated via self-assembly methods are generally periodic, and therefore cannot compete with flexible and non-periodic designs offered by lithographic methods.

In the last decade, a number of diverse biomimetic approaches have been explored for nanomaterials fabrication. The central and most promising approaches for nanotechnology have been based on (i) specificity of programmable interactions of nanoscale objects due to biomolecular recognition; (ii) assembly of structures that can direct self-assembly processes; and (iii) bio-mineralization or metallization processes. A variety of different biological systems have been suggested for the realization of biomimetic nanoassembly including viruses, DNAs, peptides and proteins. The validity of these approaches has recently been demonstrated for the assembly of semiconductor and metallic nanowires based on hybridization of DNA oligomers, assembly of the DNA functionalized particles, synthesis of DNA-based ‘nanocrystal molecules’, formation of hierarchical self-assemblies from lipid-actin complexes, and assembly of 3D DNA guided superlattices of nanoparticles.

Among the various biomolecular materials, DNA has attracted much attention due to its unique recognition capabilities, mechanical and physicochemical stability, and synthetic accessibility of practically any desired nucleotide sequences. The development of structural nucleic acid nanotechnology has been facilitated by the advancement of nucleic acid synthesis technology. For example, technology has progressed such that DNA of any desired sequence can be synthesized up to about 200 bases in a single strand. These synthetic strands of DNA can self-assemble into complex, branched structures and mechanical assemblies. The features of these assemblies can be approximately two nanometers in size, which is equivalent to the width of a DNA double helix (ALDAYE, F. A.; SLEIMAN, H. F. Journal of the American Chemical Society 129(14): 4130-4131 (2007); KUMARA, M. T.; NYKYPANCHUK, D.; SHERMAN, W. B. Nano Letters 8(7): 1971-1977 (2008); SHIH, W. M.; QUISPE, J. D.; JOYCE, G. F. Nature 427(6975):618-621 (2004); ZHANG, X. P.; YAN, H.; SHEN, Z. Y.; SEEMAN, N. C. Journal of the American Chemical Society 124(44):12940-12941 (2002)). Accordingly, DNA nanotechnology is one of the premier techniques for forming structures in the nanometer size range because of the wide variety of possible structures that can form through assemblies driven by Watson-Crick base pairing.

Recently several groups have reported assembly of nano-objects into arrays using DNA scaffolds (LE, J. D., et al, “DNA-Templated Self-Assembly of Metallic Nanocomponent Arrays on a Surface”, Nano Letters, 4(12), 2343-2347, (2004); DENG, Z. X., et al, “DNA-Encoded Self-Assembly of Gold Nanoparticles into One-Dimensional Arrays”, Angew. Chem. Int. Ed., 44, 3582-3585, (2005); ZHANG, J. P., et al, “Transparent, Conductive, and Flexible Carbon Nanotube Films and Their Application in Organic Light-Emitting Diodes”, Nano Letters, 6(2):248-251, (2006)). Various types of patterns were are capable of being formed by designing branched DNA structures. These DNA patterns have the ability to incorporate DNA binding sites for potential attachment of DNA coated nano-objects via hybridization (MIRKIN, C. A., et al, “A DNA-Based Method For Rationally Assembling Nanoparticles Into Macroscopic Materials”, Nature, 382(6592):607-609, (1996); ALIVISATOS, A. P., et al, “Organization Of Nanocrystal Molecules' using DNA” Nature, 382:609-611, (1996); MAYE, M. M., et al, “DNA-Regulated Micro- and Nanoparticle Assembly”, Small 3, 1678-1682, (2007)). However, only periodic placement of nano-objects was possible using this approach as demonstrated by the regular periodic patterns that were observed. Additional limitations of this approach include: (i) the complexity of structures and ability to incorporate various types of elements are restricted because the unit cell of periodic structures is typically small (e.g., on the order of a few nanometers to tens of nanometers); (ii) the size of uniform scaffold area is typically only a few nanometers; (iii) Magnesium ions are required to stabilize DNA scaffolds which often induce uncontrollable aggregation of DNA coated nano-objects; (iv) typically mica surfaces are required for DNA scaffold immobilization that limit a choice of materials on which structure can be created; and (v) there are technological limits with the applications and integration with other fabrication techniques because the placement or orientation of scaffold is difficult to control.

Fabrication of arbitrary shapes has been successfully demonstrated by folding genetic single stranded (ss) DNA into particular predesigned shapes, known as DNA origami (ROTHEMUND, P. W. K., “Folding DNA to create nanoscale shapes and pattern”, Nature, 440:297, (2006)). However, using this approach for positioning particles is somewhat restricted because of factors (ii-v) as discussed above. Additionally, the size of a DNA origami structure is restricted to a few hundred nanometers because ss-DNA significantly limit design and scalability of the system. Other recently developed approaches for 3D ordering of nano-objects using DNA have been limited to periodic structures as well (NYKYPANCHUK, D., et al, “DNA-guided crystallization of colloidal nanoparticles”, Nature, 451(7178):542-552, (2008)).

Thus, there is a need for creating an arbitrary assembly of nano-objects on arrays that overcome the limitations known in the art.

### SUMMARY

The present invention is directed to nanoscale fabrication of broad classes of nano-materials with application in electronics, energy conversion, bio-sensing, and others. Specifically, the present invention is directed to arbitrary, that is periodic and non-periodic, assembly of nano-objects on 1D and 2D arrays. The present invention utilizes self-organization properties of nanoscale bio-encoded building blocks, programmability of biomolecular interactions, and simple processing techniques for providing arbitrary by-design fabrication capability. Moreover, the present invention allows for an integration of a large number of nano-components and their types in unified well-defined systems.

The present invention is applicable for fabrication of 1D and 2D structures of various by-design placements of nano-objects of multiple types, including metal, semiconducting and organic nano-objects. The present invention provides nanometer level precision in a registration of nano-object on a pre-designed site and allow to create structures with sizes of tens microns or larger.

In one embodiment, the present invention provides a one dimensional matrix that directs the organization of nano-objects onto row DNAs. Row DNAs are created by deposition and attachment of double stranded lithographic DNA onto a surface through an anchoring point. This allows for a by-design fabrication of an arbitrary matrix of individually encoded sites on lithographic ds-DNA. Using specific intercalators which bind to pre-determined regions of lithographic ds-DNA, encoded nano-objects recognize their position with nm-level accuracy via self-assembly. The present invention provides versatility of integration of multiple types of objects over at least tens of microns

In another embodiment, the present invention provides two dimensional matrices that direct organization of nano-objects onto column and row DNAs. A number of DNA anchoring points on column lithographic DNA provide specific sites for attachment of row DNA. This allows for a by-design fabrication of arbitrary matrix of individually encoded sites on lithographic ds-DNA rows. Using specific intercalators which binds to pre-determined regions of ds-DNA, encoded nano-objects recognize their position on 2D matrix with nm-level accuracy via self-assembly. The present invention provides versatility of integration of multiple types of objects over at least tens of microns.

The versatility of integration by the present invention is difficult or nearly impossible to achieve today by any other methods. The present invention can also be combined with existing optical lithography methods, which can enable the fabrication of large scale features tens of microns in size. The present invention naturally incorporates 1D arbitrary assembly and ultimately can be extended into 3D.

### BRIEF DESCRIPTION OF THE DRAWINGS

FIG. 1A is an illustration of PNA-directed AuNP assembly according to an embodiment of the present invention.

FIG. 1B provides sequences used for PNA-directed AuNP assembly according to an embodiment of the present invention.

FIG. 2A is a TEM micrograph of DNA functionalized AuNPs according to an embodiment of the present invention.

FIG. 2B provides DLS measurements of AuNPs and DNA functionalized AuNPs according to an embodiment of the present invention.

FIG. 3A is a representative TEM micrograph of an aggregation of Au nanoparticles based on PNA invasion of dsDNA according to an embodiment of the present invention.

FIG. 3B is a statistical analysis of the TEM micrograph shown in FIG. 3A.

FIG. 3C provides DLS measurements of Dₕ (hydrodynamic diameter) of single particles (control, red) and assembled aggregates (black) according to FIG. 3A.

FIG. 4A is a representative TEM micrograph showing aggregation of Au nanoparticles based on PNA invasion of dsDNA at 4° C. and the inset TEM micrograph is for the control sample, in which no PNA-DNA chimera is added according to an embodiment of the present invention.

FIG. 4B is a statistical analysis of the TEM micrograph shown in FIG. 4A.

FIG. 4C provides DLS measurements of Dₕ (hydrodynamic diameter) of single particles (control, red) and assembled aggregates (black) according to FIG. 4A.

FIG. 5A is an illustration showing A-AuNPs mixed with A′ Complementary DNA, A″-PNA-DNA-B′, and Cy3-DNA B (complementary to DNA-B′) according to an embodiment of the present invention.

FIG. 5B is an analysis of the PNA to NP binding according to FIG. 5A.

FIG. 6A is a UV-vis melting curve of A″-PNA-DNA-B′ and DNA-B complementary according to an embodiment of the present invention.

FIG. 6B is a Dynamic Light Scattering (DLS) melting curve of DNA A-AuNP, DNA B-AuNP, A′-DNA complementary, and A″-PNA-DNA-B′ according to an embodiment of the present invention.

FIG. 7 is an illustration of PNA-directed AuNP assembly along a dsDNA according to an embodiment of the present invention.

FIG. 8A is a representative TEM micrograph of assembled nanoclusters based on PNA invasion of dsDNA according to the schematic in FIG. 7 and an embodiment of the present invention.

FIG. 8B illustrates three possible configurations of assembled trimers along a dsDNA on a flat surface (top: schematic; bottom: TEM images) based on the TEM micrograph depicted in FIG. 8A.

FIG. 8C provides statistical analysis based on FIG. 8A.

FIG. 8D provides DLS measurements of Dₕ of single particles (control, red) and assembled clusters (black) based on FIG. 8A.

FIG. 9 is an illustration showing deposition and attachment of double stranded lithographic DNA according to an embodiment of the present invention.

FIG. 10 is an illustration showing binding of intercalators to double stranded DNA with free attachment sites: ss-PNA-DNA chimeras invasion of ds-DNA at specific locations according to an embodiment of the present invention.

FIG. 11 is an illustration showing nano-objects that bind to specific DNA locations via recognition of free attachment sites of bound intercalators according to an embodiment of the present invention.

FIG. 12, top is an illustration showing fabrication of a DNA array using multiple anchoring points according to an embodiment of the present invention.

FIG. 12, bottom, is an illustration showing assembly of nano-objects array on DNA/intercalator lithographic array according to an embodiment of the present invention.

FIG. 13 is an illustration showing deposition fixation of initial column DNA (Y-DNA) with encoded regions for intercalators placements according to an embodiment of the present invention.

FIG. 14 is an illustration showing fabrication of 2D arbitrary matrix of encoded sites according to an embodiment of the present invention.

FIG. 15 is an illustration showing fabrication of nano-objects array using 2D arbitrary matrix of encoded object-recognizable sites according to an embodiment of the present invention.

### DETAILED DESCRIPTION

In the interest of clarity, in describing the invention, the following terms and acronyms are defined as provided below.

ACRONYMS:
DNA: Deoxyribonucleic acid
RNA: Ribonucleic acid
PNA: Protein Nucleic Acid
NP: Nanoparticle
AuNP: Gold Nanoparticle
1D: One-Dimensional
2D: Two-Dimensional
ds: Double Stranded
ss: Single Stranded

### DEFINITIONS

Intercalator: A molecule having one end that is capable of binding to a specific site on a DNA, matrix, or array (intercalator binding site) and second end that serves as an attachment site for a bio-encoded nano-object (nano-object binding site).
Lithographic DNA: Double stranded DNA with specifically designed regions for intercalator binding (intercalator binding sites).
Arbitrary assembly: Periodic and non-periodic by-design fabrication of nanoscale bio-encoded building blocks.
Periodic: Occurring at regularly spaced intervals.
Non-periodic: Occurring at non-regularly spaced intervals.
Nanoparticle: Any manufactured, naturally, or chemically produced structure or particle with nanometer-scale dimensions (i.e., 1 to 100 nm).
Matrix: A total population of encoded sites for given polymers or biopolymers with well defined encoded binding sites (e.g., nucleic acids, peptides, polymer chains with chemically active groups). The same matrix might be used for making different arrays depending what sites (on a matrix) are chosen.
Array: A structure or architecture of compounds in the form of an organized matrix that contains a specifically encoded sites for binding of correspondingly encoded particles. The array is used to make specific arbitrary assembly.
Row DNA: DNA that aligns on a surface along an x-axis.
Column DNA: DNA that aligns on a surface along a y-axis.

The present invention is directed to a method for the by-design fabrication of arbitrary, non-periodic and periodic, 1D and 2D arrays of nano-objects of multiple types and compositions. Any arbitrary 1D or 2D structure (array) can be represented as a matrix with nano-objects positioned in predesigned sites, which positions are determined by their horizontal (X) and vertical (Y) coordinates, and each position on the matrix possesses some chemical, electrical, biological or other functionality. The present invention can be used to create any arbitrary 1D or 2D architecture from nano-objects through the fabrication of a highly specific matrix.

The arrays and/or matrices of the present invention can be attached to any surface that can bind arrays and/or matrices without inhibiting or interfering with the array and/or matrix structure. For example, the surface can be a solid surface, a membrane, microscopic beads, a film, or any other type of surface capable of binding a matrix and/or array. The surface can be composed of any material, for example, glass, silicon, silica, mica, metal, plastic, Polyvinylidene Fluoride (PVDF), nitrocellulose, semiconductor, graphene or combinations thereof. In a preferred embodiment, the surface is a solid support made of silicon.

The matrix and/or array of the present invention can be any chemical or compound that is capable of binding to a surface and capable of binding to intercalators in a periodic and non-periodic manner. For example, the matrix and/or array may be comprised of small molecules or macromolecules used alone or in combination. Examples of macromolecules that can be used include nucleic acids (e.g., DNA, RNA, and/or combinations thereof); amino acids (e.g., traditional and modified amino acids, peptides, proteins, amino acid-nucleic acid hybrids, and/or combinations thereof); carbohydrates (e.g., monosaccharides, polysaccharides, oligosaccharides, and/or combinations thereof); or lipids (e.g., fatty acids, glycerolipids, glycerophospholipids, sphingolipids, sterol lipids, prenol lipids, saccharolipids, polyketides, fats, waxes, sterols, fat-soluble vitamins, monoglycerides, diglycerides, phospholipids, and/or combinations thereof).

In some embodiments of the present invention, the matrix and/or array is prepared using nucleic acids. In a preferred embodiment, the matrix and/or array is prepared using DNA. Naturally occurring and/or genetically engineered DNAs of any sequence can be used to encode the structure of the matrix and/or array. In a more preferred embodiment, the DNA is lithographic ds-DNA, that is, linear, ds-DNA designed with pre-determined sequences that provide specifically encoded locations for attachment of intercalators. The specifically designed regions for intercalator binding can be arbitrary, that is, periodic (separated at regular intervals) or non-periodic (separated by non-regular intervals).

The structure of the matrix and/or array is not limited to any pattern, shape, or size. In some embodiments, the structure of the matrix and/or array is essentially linear or one-dimensional. In other embodiments, the structure of the matrix and/or array is non-linear or two-dimensional. In yet other embodiments, the structure of the matrix and/or array is three-dimensional.

In a preferred embodiment, when the matrix and/or array is 2D, the matrix and/or array is prepared by assembling lithographic DNA into an XxY array containing one or more than one row and one or more than one column, as illustrated further below. Alternatively, lithographic DNA can be arrange in non-linear or non-rectangular patterns, for example, in circular-like, sinusoid-like, etc.

The matrix and/or array can be attached to the surface, as described above, by a number of different specific or non-specific methods. For example, the matrix and/or array can be attached to the surface by covalent bonds, non-covalent bonds, electrostatic interactions, protein-protein interaction, DNA-DNA interaction, protein-nucleic acid interaction, protein substrate interaction, and the like. In a preferred embodiment, the DNA is bound through an anchoring point via DNA-hybridization or biotin-streptavidin interaction.

Intercalators are molecules or compounds that have at least two ends. One end of the intercalator is capable of recognizing and binding to specific locations on a matrix and/or array. A second end of the intercalator is capable of attaching to a bio-encoded nano-object. Intercalators of the present invention can be any chemical or compound that is capable of binding to a matrix and/or array on one end and to a nano-object on another end. For example, the intercalators may be comprised of small molecules or macromolecules used alone or in combination. Examples of macromolecules that can be used include nucleic acids (e.g., DNA, RNA, and/or combinations thereof); amino acids (e.g., traditional and modified amino acids, peptides, proteins, amino acid-nucleic acid hybrids, and/or combinations thereof); carbohydrates (e.g., monosaccharides, polysaccharides, oligosaccharides, and/or combinations thereof); or lipids (e.g., fatty acids, glycerolipids, glycerophospholipids, sphingolipids, sterol lipids, prenol lipids, saccharolipids, polyketides, fats, waxes, sterols, fat-soluble vitamins, monoglycerides, diglycerides, phospholipids, and/or combinations thereof). In a preferred embodiment, the intercalator is a protein nucleic acid (PNA).

Nano-objects of the present invention are not limited to any type, shape, or size. Examples of nano-objects include small and macromolecules used alone or in combination. Examples of macromolecules that can be used include nanoparticles, nucleic acids (e.g., DNA, RNA, and/or combinations thereof); amino acids (e.g., traditional and modified amino acids, peptides, proteins, amino acid-nucleic acid hybrids, and/or combinations thereof); carbohydrates (e.g., monosaccharides, polysaccharides, oligosaccharides, and/or combinations thereof); or lipids (e.g., fatty acids, glycerolipids, glycerophospholipids, sphingolipids, sterol lipids, prenol lipids, saccharolipids, polyketides, fats, waxes, sterols, fat-soluble vitamins, monoglycerides, diglycerides, phospholipids, and/or combinations thereof).

In a preferred embodiment, the nano-object is a nanoparticle (NP). Examples of nanoparticles include metallic (e.g., gold, silver, platinum), semiconductive (e.g., CdSe, CdTe, CdSeZnS), or magnetic (e.g., Fe₂O₃, FePt) nanoparticles. Additionally, NPs can be of any shape, such as spherical, rod-shaped, icosahedral, planar, tubular, etc. As used herein, unless otherwise noted, “particle” should be construed to include micro-objects (including microspheres, microrods, etc.) and nano-objects (fullerenes, quantum dots, nanorods, nanotubes, etc.). In one embodiment the nanoparticle is metallic. In a specific embodiment, the nanoparticle is a gold nanoparticle (AuNP).

### EXAMPLES

The following examples and references to the figures should not be considered limiting in any way. General materials and techniques are described; however, it should be understood that variants of the disclosed materials, sequences, and/or methods have been considered by the inventors and are deemed as part of the invention.

### Materials

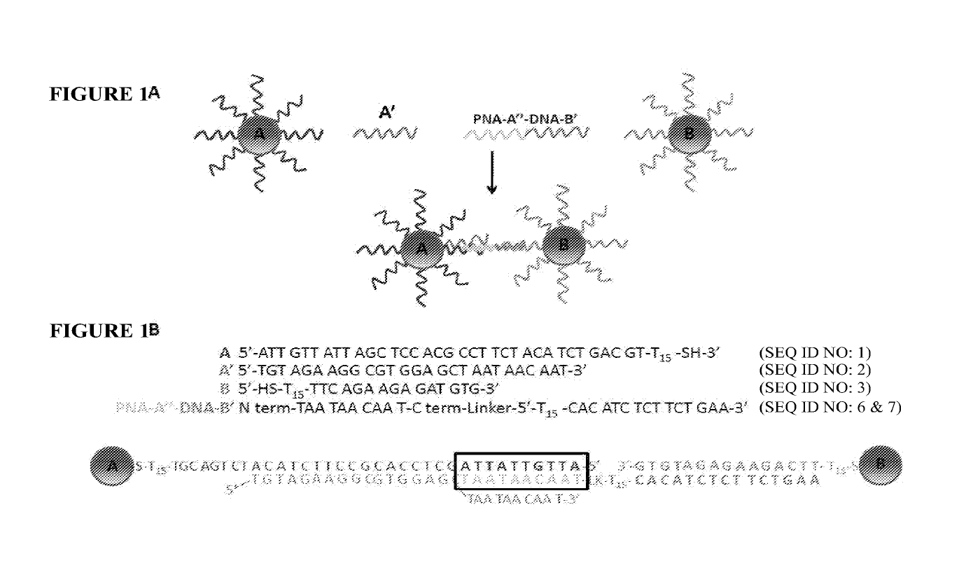

DNA oligonucleotides were purchased from Integrated DNA Technologies, Inc. (www.idtdna.com) as lyophilized powders. Unmodified and thiolated oligonucleotides were purified by gel filtration chromatography. Sequences for the DNA strands, which are also identified in FIG. 1 were:

A:
(SEQ ID NO: 1)
5′-ATT GTT ATT AGC TCC ACG CCT TCT ACA TCT GAC GT-
T15-SH-3′
A′:
(SEQ ID NO: 2)
5′-TGT AGA AGG CGT GGA GCT AAT AAC AAT-3′
B:
(SEQ ID NO: 3)
5′-HS-T1S-TTC AGA AGA GAT GTG-3′
200-bp ssDNA A:
(SEQ ID NO: 4)
5′-TCC GCA AGC TGG CCC TCA CTT CAA CGC ATT
ATT GTT AAT CTT CCA ATG GGC CAC CTA CCG TAG ACA
CGG ACT CTC TAC GCG TTA TGC CTC AGC
ATA TTA TTG TTA CTG CGG GAC ATA CGA TAG AGC TTT
GCT AAA ATA AGT CCC TGC CTT TCC ACC AAT AGA
AAT TAT TGT TAC GTA GCC AAT CGA CGT ATT TGG TAC
GT-3′
200-bp ssDNA A':
(SEQ ID NO: 5)
5′-ACG TAC CAA ATA CGT CGA TTG GCT ACG
TAA CAA TAA TTT CTA TTG GTG GAA AGG CAG GGA CTT
ATT TTA GCA AAG CTC TAT CGT ATG TCC CGC
AGT AAC AAT AAT ATG CTG AGG CAT AAC GCG TAG AGA
GTC CGT GTC TAC GGT AGG TGG CCCATT GGA AGA
TTA ACA ATA ATG CGT TGA AGT GAG GGC CAGCTT GCG GA-
3′

Three identical anchoring positions are underlined, which can be “invaded” by the PNA part of the PNA-DNA chimera.

PNA-DNA chimeras were synthesized and purchased from Bio-Synthesis Inc. as lyophilized powders. The chimeras can be further purified by HPLC techniques known in the art. Sequences for the chimeras were:

(15 bp-DNA Chimera): A″-PNA-DNA-B′:
(SEQ ID NOs: 6 & 7)
5′-TAA TAA CAA T-linker-T15-CAC ATC TCT TCT GAA-3′
(10-bp DNA Chimera): A″-PNA-DNA-B₂′:
(SEQ ID NO: 6 & 8)
5′-TAA TAA CAA-Linker-CAC ATC TCT T

The PNA is underlined and is written from N-C and the DNA is written from 5′-3′. The linker is: cysteine-SMCC-C6 amino.

### Au Nanoparticle Synthesis

10-nm Au nanoparticles were synthesized through a classic citrate reduction method with slight modifications. Briefly, 1 mM HAuCl₄ aqueous solution was first heated to boil for 20-30 minutes. Subsequently, 10 mL of trisodium citrate solution with a concentration of 38 nM was added to the above solution. The reaction was allowed to continue until the initial color changed to red, and quenched by deionized water. After the Au nanoparticle solution cooled to room temperature, it was stored in a glass bottle at ambient condition for further functionalized with DNA. The particle size was examined by DLS and TEM and the concentration was determined through UV-vis absorption at λ=519 nm with an extinction coefficient of 1.0×10⁸ L·mole⁻¹ cm⁻¹.

### Functionalization of Au Nanoparticles

The thiol functionality of the DNA was deprotected by the addition of 0.1 M dithiothretol (DTT) for at least 2 hrs on ice prior to DNA loading (typically, 10-11 OD of concentrated DNA; 200 μl of DTT). The deprotected DNA solutions were purified using desalting NAP-5 columns (Sephadex G-25, Amersham Biosciences). Au nanoparticles were functionalized with deprotected thiol-oligonucleotides following methods for high DNA coverage reported by Mirkin and co-workers (Mirkin, C. A., et al., Nature, 1996. 382(6592): p. 607-609; which is incorporated herein by reference). In a typical experiment with 10 nm gold nanoparticles, an aliquot (1-50 μl) of a purified DNA 50-300 μM solution was added to a 1 mL aliquot of gold particles (10-30 nM). The ssDNA and particle solutions were incubated at room temperature in a non buffered solution for at least 3 hr before adding phosphate buffer to bring its concentration to 10 mM (pH=7.4). The solution was left to anneal at 25° C. for 4 hr before the addition of NaCl (0.025M). The salt concentration was then increased gradually from 0.025 to 0.3 M NaCl over 24 hr, and left to anneal for an additional 24 hr at 0.3M. The excess DNA next was removed from the solutions by centrifugation for 30 minutes at 4,500 g.

### Characterization of Aggregates and Trimers.

Dynamic Light Scattering (DLS):

DLS measurements were performed on a Malvern Zetasizer ZS instrument. The instrument was equipped with 1 633 nm laser source and a backscattering detector at 173°.

Transmission Electron Microscopy (TEM):

TEM micrographs of DNA-functionalized Au NPs and assembled aggregates and nanoclusters were collected using a JEOL 1300 transmission electron microscope operated at 120 kV. Samples were prepared by placing a droplet of the aqueous solution onto a 400-mesh carbon-coated copper grid, followed by drying at room temperature for overnight before imaging.

### Example 1

### PNA-Directed Assembly of Aggregates

A specific PNA-DNA chimera was used to direct the formation of macroscopic aggregates of DNA functionalized AuNPs, as depicted in FIG. 1A. Gold nanoparticles, 10 nm in diameter, were functionalized with two types of non-complementary single-stranded (ss) DNA, A and B (A-AuNPs and B-AuNPs), respectively (described above and shown in FIG. 1B). The molar concentration of AuNP probes were measured by UV-vis spectroscopy (molar extinction coefficient 1.0×10⁸ M⁻¹ cm⁻¹ at 524 nm). An equimolar concentration of DNA-functionalized AuNPs (A-DNA AuNPs and B-DNA AuNPs) were mixed with a 10-fold excess of A′-DNA, and A″-PNA-DNA-B′. The solution was heated to 65° C. for 10 minutes, and slowly cooled to room temperature in 0.1 M PBS (0.1M sodium chloride, 10 mM sodium phosphate buffer, pH 7.0). The aggregates were characterized without further purification.

In this process, a tertiary complex is formed between the A-DNA sequences on the A-AuNPs, complementary A′-DNA, and the A″-PNA-DNA-B′ chimera. The oligonucleotides on A-AuNPs partially hybridized to A′ through a 27-base-pair (bp) A-A′ DNA-DNA sequence recognition. The higher affinity of PNA to SSDNA due to the lack of charge of the PNA backbone, allow the A″-PNA sequence of the A″-PNA-DNA-B′ chimera to “invade” and form a 10-bp duplex at the end of the A-DNA sequence immobilized on the AuNP. Meanwhile, the B′ strands in the A″-PNA-DNA-B′ chimera hybridize to B-AuNPs through a 15-bp B-B′ DNA-DNA sequence recognition. In such a fashion, nanoparticle aggregates are formed between non-complementary A-AuNPs and B-AuNPs through PNA-directed assembly.

Aggregation of AuNPs Based on PNA Invasion of dsDNA

PNA-directed aggregation of non-complementary AuNPs was monitored using transmission electron microscopy (TEM) and dynamic light scattering (DLS) without any further purification.

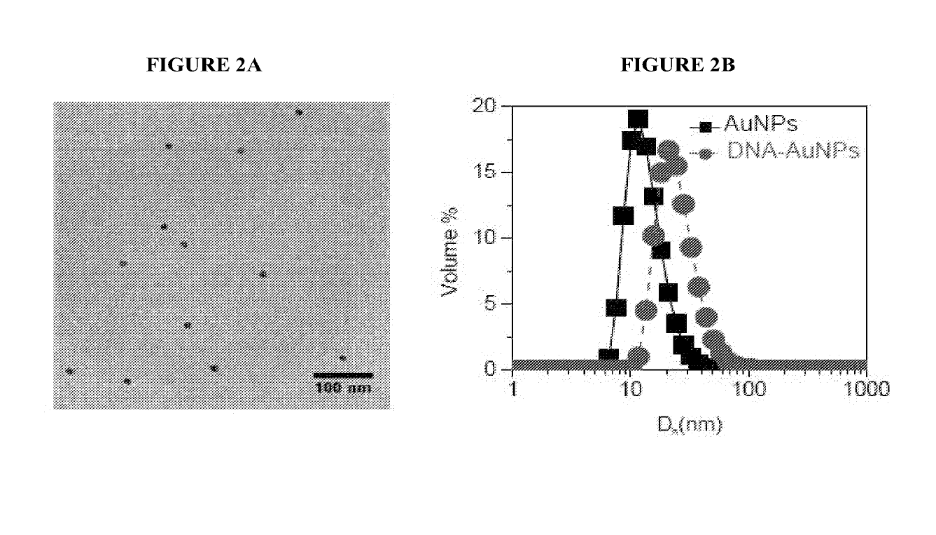

As an initial control, AuNPs and DNA functionalized AuNPs were evaluated in the absence of a linker. FIG. 2A shows a representative TEM micrograph of DNA functionalized AuNPs. The TEM shows single nanoparticles that do not assemble to form clusters. DLS profiles characterizing the volume-averaged hydrodynamic diameter (Dh) population of the DNA functionalized AuNPs and control sample (non-functionalized AuNPs) are shown in FIG. 2B. The DNA functionalized AuNPs exhibit a single population at Dh≈25 nm, which is shifted in comparison to the non-functionalized AuNPs.

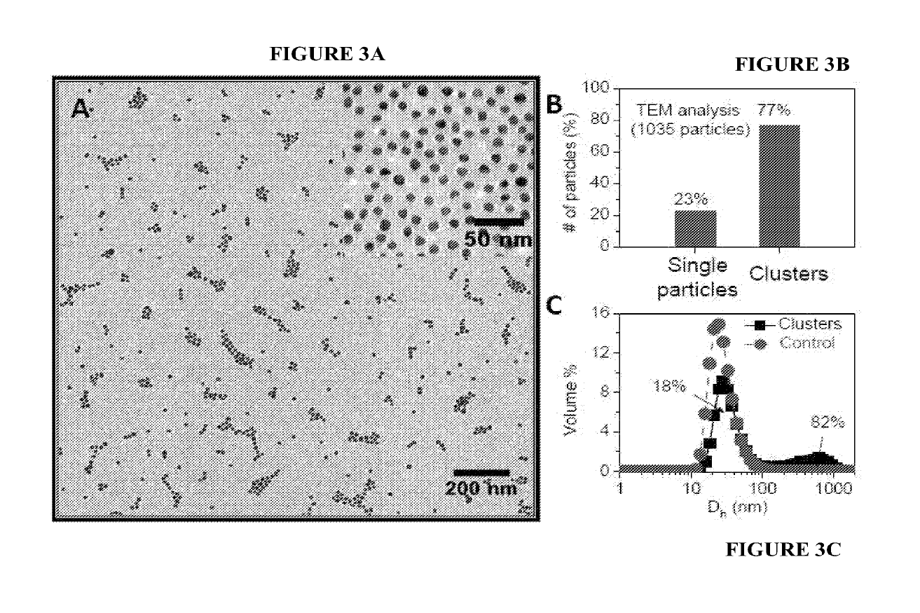

FIG. 3A shows a representative TEM micrograph, illustrating the formation of nanoparticle clusters by an embodiment of the present invention. Specifically, a linker was added to AuNPs functionalized with DNA according to the method described above, and in FIG. 1 (Similar to FIG. 2A, a control was also conducted without using the PNA-DNA chimera linker (FIG. 3A inset) which again showed no connection between nanoparticles). Statistical analysis based on the TEM observations in FIG. 3A revealed that ˜77% of nanoparticles were assembled into larger aggregates (n=1035 particles) (FIG. 3B). DLS profiles characterizing the volume-averaged hydrodynamic diameter (Dh) population of the assembled aggregates and control sample are shown in FIG. 3C. The control sample exhibits a single population at Dh≈25 nm, similar to the DNA-functionalized AuNPs (FIG. 2B). However, the sample using the PNA “invasion” approach shows an additional population at Dh=100-2000 nm, suggesting the existence of the larger-scale aggregates. Moreover, a statistical analysis based on the DLS profile reveals a yield of 82% of assembled aggregates, agreeing well with the TEM analysis.

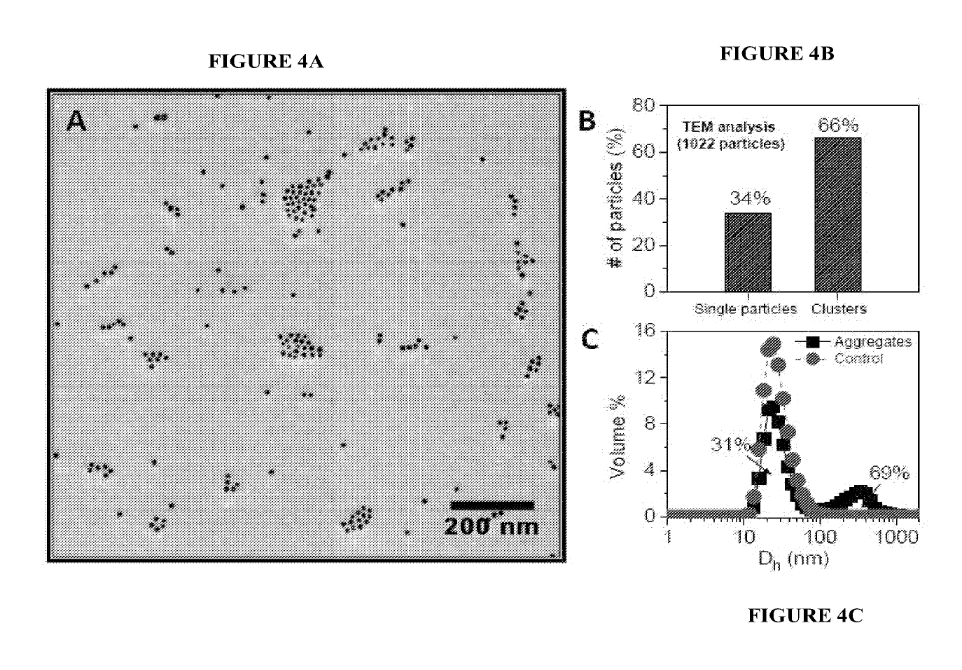

A similar experiment was also conducted using a PNA-DNA chimera with 10-bp PNA and 10-bp DNA (A″-PNA-DNA-B₂′) at 4° C. to compare with the experiments performed at room temperature (the PNA-DNA chimera used in the room temperature experiments has 10-bp PNA and 15-bp DNA). Nanoparticles also assembled into large aggregates in this scenario (FIG. 4).

### Quantitation of Hybridized PNA-DNA Chimeras

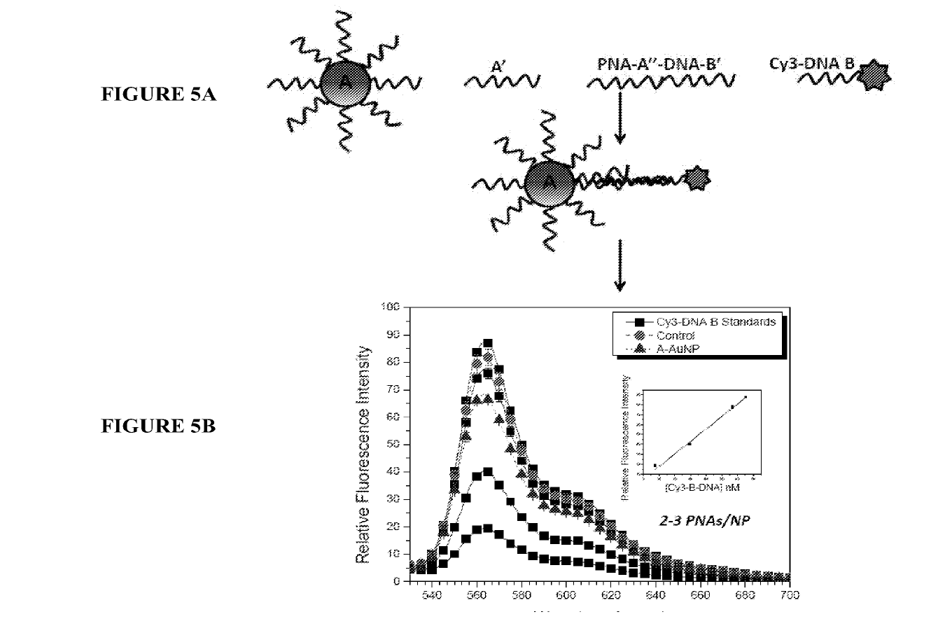

The extent of PNA-DNA chimera binding to A-AuNPs was determined according to the method outlined in (FIG. 5). Similar to the PNA-directed aggregation experiments, A-AuNPs were mixed with a 10-fold excess of complementary A′-DNA, A″-PNA-DNA-B′ chimera, and Cy3-DNA-B was used to replace B-AuNPs. The solution was heated to 65° C. for 10 minutes and slowly cooled to room temperature in 0.1M PBS. Unhybridized Cy3-DNA-B was removed by centrifugation, and amount of hybridized DNA was determined by fluorescence spectroscopy using a Varian Fluorimeter. The change of fluorescence of the supernatant shows that approximately 2-3 PNA-DNA chimeras bind per nanoparticle (FIG. 5). The relative low efficiency of PNA “invasion” indicates a low accessibility to the A-AuNP surface, and supports the formation of the smaller macroscopic aggregates observed in TEM images and DLS data.

### Melting Profiles

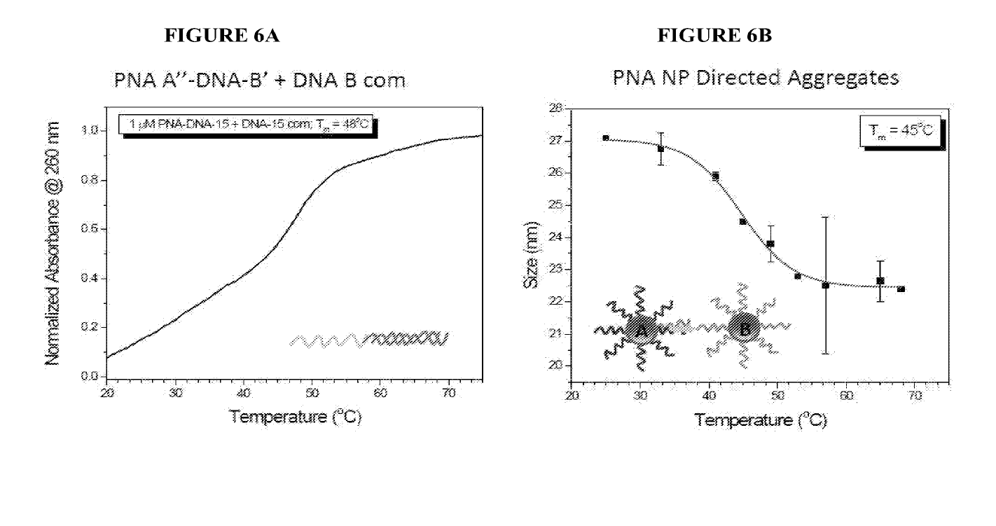

Duplex DNA structures formed between target DNA and DNA on nanoparticles typically exhibit sharp melting profiles and increased melting temperatures in aggregate assemblies compared to single complementary DNA strands. The UV melting curve of a 1 μM solution of 15-bp DNA duplex formed between A″-PNA-DNA-B′ and B-DNA shows a broad melting curve and the duplex melts with a Tm=48° C. (FIG. 6A). Temperature dependent dynamic light scattering was used to determine the melting transition of the PNA-directed aggregate assemblies (FIG. 6B). Here, the concentration of DNA is 200-fold less than in UV-vis melting experiments. The melting transition observed is sharper than that observed for the duplex in the absence of nanoparticles, however, the melting temperature of the nanoparticle-linked 15-bp DNA duplex (Tm=45° C.), is in agreement with that obtained for the DNA duplex alone. The similar melting temperatures indicate the temperature-dependent change in size observed is due to thermal dissociation of the PNA-directed aggregates.

This example demonstrates a new strategy to assemble DNA-functionalized nanoparticles by the concept of PNA “invasion” of dsDNA by specifically polymerizing dsDNA-modified AuNPs into aggregates.

### Example 2

### PNA-Directed AuNP Assembly on dsDNA (PNA “Invasion” for Trimer Formation)

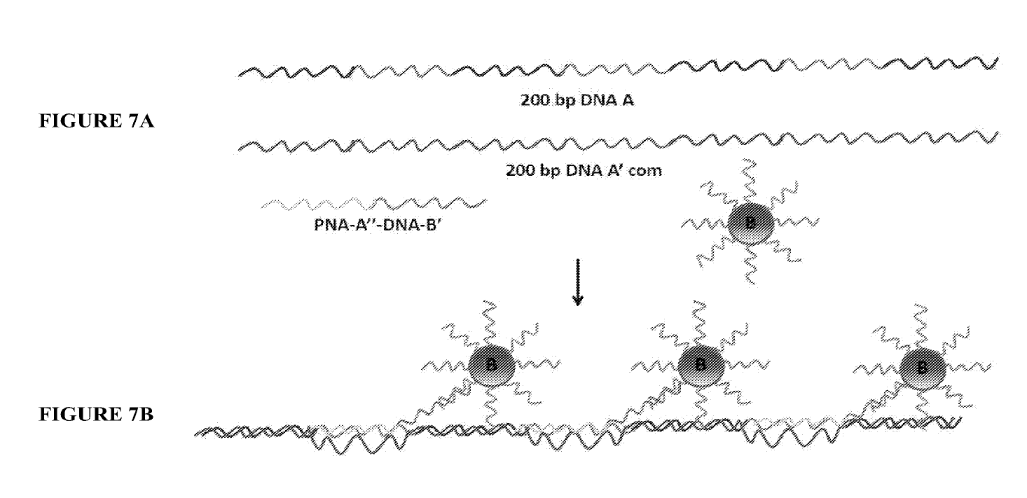

The PNA “invasion” strategy was also used to assemble AuNPs into well-defined nanoclusters along a dsDNA template which is shown in (FIG. 7). A 200-bp ds-DNA was designed that contains three identical 10-bp fragments which are complementary to the 10-bp PNA part (A″) of the PNA-DNA chimera. 10-nm AuNPs were functionalized with a ssDNA B (B-AuNPs) that is complementary to the 15-bp DNA part (B′) of the PNA-DNA chimera. A mixture was prepared containing 200-bp ssDNA A, 200-bp ssDNA A′, A″-PNA-DNA-B′, and B-AuNPs in a molar ratio of 1:1:3:3 in 0.1 M PBS. The mixture was then heated to 65° C. for 10 minutes, and cooled to room temperature for overnight. In this process, the PNA-DNA chimera “invaded” the 200-bp dsDNA duplex at the designed locations to create three anchors, and then the DNA-functionalized AuNPs can recognize these anchors on the dsDNA duplex through DNA-DNA base-pairing hybridization. In this manner, nanoparticle trimers assembled along the dsDNA duplex.

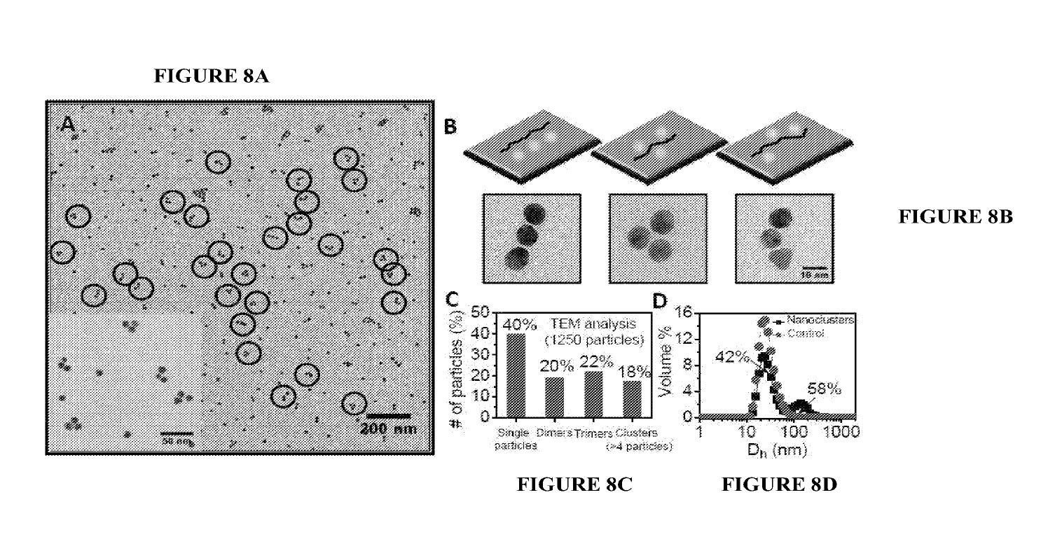

The assembled nanoparticle trimers were characterized by TEM and DLS. The TEM image in FIG. 8A reveals a mixture of single particles, dimers, trimers, and larger clusters from the sample. The circles around the clusters indicate assembled trimers. FIG. 8B illustrates three possible configurations of assembled trimers along a dsDNA on a flat surface (top: schematic; bottom: TEM images). A statistical analysis based on the TEM observation (FIG. 8C) suggests that the sample contained 40% of single nanoparticles, 20% of dimers, 22% of trimers and 18% of larger clusters (4-10 particles). DLS profiles of the assembled and control (without adding PNA-DNA chimera) solutions in FIG. 8D also demonstrate the formation of nanoparticle clusters by the PNA “invasion”. A statistical analysis on the DLS result suggests a yield of 58% nanoparticle clusters, which was consistent with the TEM analysis (60% in total for dimers, trimers, and larger clusters).

The impurities that result from the design can be attributed to several factors. The formation of larger clusters may be due to the fact that after linked to the PNA-DNA anchor on one dsDNA duplex, the surface of the DNA-functionalized AuNPs has not been passivated so that they can also hybridize with other dsDNA duplexes “invaded” by the PNA-DNA chimera. Therefore, larger nanoparticle clusters are formed using the present invention. The presence of single nanoparticles and dimers, could be due to the “invasion” efficiency of the PNA-DNA chimera into the 200-bp dsDNA duplex which were demonstrated in Example 1.

This example demonstrates a new strategy to assemble DNA-functionalized nanoparticles by the concept of PNA “invasion” of dsDNA by specifically organizing ssDNA-functionalized AuNPs along dsDNA duplex.

### Example 3

### Formation of an Individual Row of a 1D Array

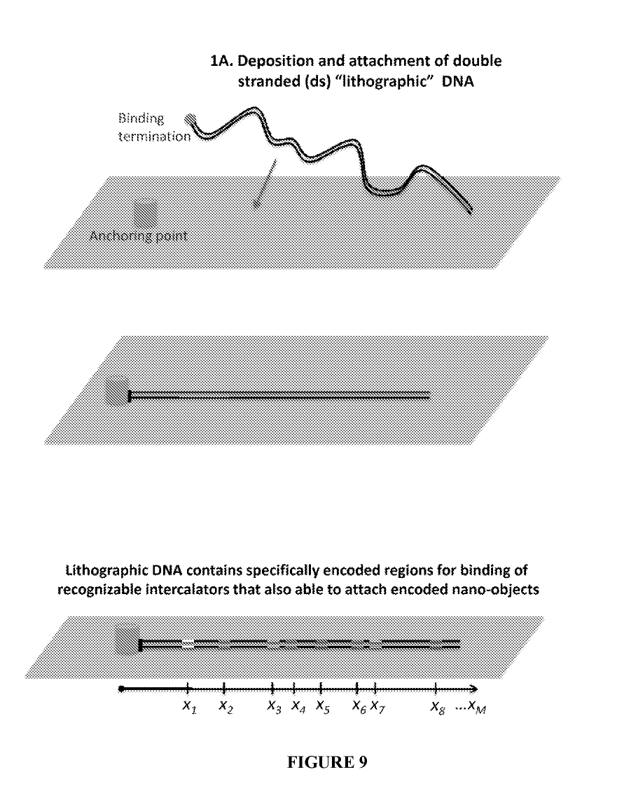

An individual row of a matrix can be fabricated in the manner shown in FIGS. 9-11 using a lithographic DNA. The steps described can be performed in any order. In a preferred embodiment, the steps are performed as set forth below.

First, ds-DNA is deposited on a surface containing an anchoring point (FIG. 9). The anchoring point can include any fabricated nano-structure, nanoparticle, or surface feature capable of binding to a DNA end. The DNA/anchoring point binding can be non-specific (thiol, silaine, etc.) or specific (e.g., DNA-hybridization, biotin-streptavidin, etc). Lithographic DNA should be designed or chosen from natural or genetic material so that the DNA sequences are known. Specific pre-determined DNA regions, called intercalator binding sites, are located along the lithographic ds-DNA for nano-object attachment (FIG. 9, bottom panel, locations identified as X₁, X₂, etc.). The specific uniqueness of each intercalator binding site is determined by DNA base pairs (bp) sequence and nucleotide length. All specific intercalator binding sites can be pre-determined and encoded via by sequences. The length of the intercalator binding sites can be any length that allows efficient intercalator binding. In some embodiments the intercalator binding site comprises 12-15 bp, which provides a robust encoding and sufficient thermal stability for intercalator/nano-object attachments.

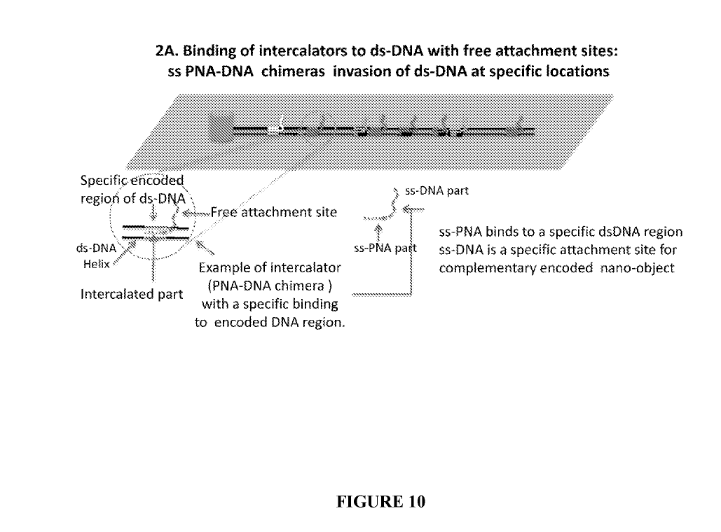

In the next step (FIG. 10), intercalators with a specific recognition to the intercalator binding sites on the matrix are added. The intercalators are capable of recognizing and specifically binding to the intercalator binding sites. The intercalators bind to the matrix at one end, and the unbound end is free to serve as an encoded recognizable attachment site for complementary encoded nano-object.

In some embodiments, the intercalator is a protein, DNA, or RNA. In preferred embodiments, the intercalator is a single stranded peptide nucleic acid (PNA) chain or a PNA-DNA chimera. A ss-PNA molecule can specifically interact with ss-DNA using Watson-Crick base pairing. The absence of charge on ss-PNA peptide backbone results in a stronger interaction between complementary sequences of ss-DNA and ss-PNA compared to ss-DNA/ss-DNA case. Single stranded PNA has the ability to interact with ds-DNA, which results in a local de-hybridization and PNA intercalation. This phenomenon is known as PNA invasion (LOHSE, J., et al, “Double duplex invasion by peptide nucleic acid: A general principle for sequence-specific targeting of double-stranded DNA”, PNAS, 96(21):11804-11808, (1999)). When a PNA-DNA chimera is added to a lithographic ds-DNA, the PNA end will bind to the intercalation binding site and the ss-DNA end will serve as a recognition site for nano-object containing a complementary functionalized strand.

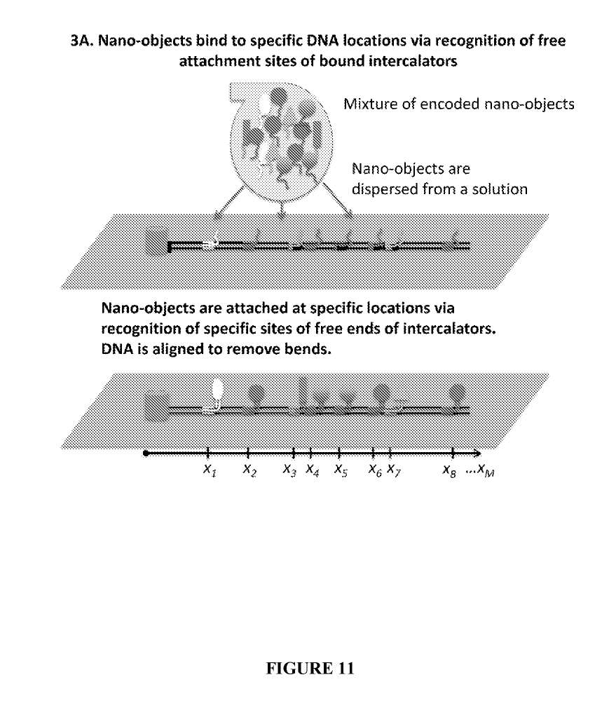

In the next step (FIG. 11), various types of nano-objects that encode nucleic acid strands complementary to the nano-object binding site on the intercalator are added. The nano-objects then recognize and specifically interact with the nano-object binding site on the intercalator, which results in a self-assembly of a 1D structure according to the instruction provided by a lithographic DNA and intercalators. Alternatively, intercalators can be directly embedded with or bound to nano-objects. A design or choice of specific sites on DNA allows for arbitrary placement of various types of nano-objects on a DNA row through the intercalators.

The accuracy of nano-object positioning can be determined by a base-pair formation and by nucleic acid chain flexibility at the attachment site. Base-pairs have a fraction of nanometer of co-localization precision, while chain flexibility can be minimized to several bases. Together this will provide 1-2 nm precision of positioning with minimum distances between sites on an order of 2-5 nm. The minimum distance between sites is determined by the length of PNA-DNA invasion region. The use of other, stronger binding intercalators may allow reducing minimum site-site separation to 1-2 nm. The use of designed and genetic DNA allows for a precise positioning of nano-objects at least on the scale of tens of microns, which allow for assembly of thousands of objects in one row. The distance between nano-objects can be between about 100 nm to 1 mm, preferably between about 1 to about 100 microns, and more preferably between about 3 to about 20 microns.

The DNA can be aligned in order to minimize its large scale bends. The DNA straightening step can be performed at any stage. In a preferred embodiment, the DNA is straightened after all nano-objects are assembled on lithographic ds-DNA.

The DNA can be straightened using a fluid flow, an electric field, or by optical tweezers (ALLEMAND, J. F., et al, “Stretching DNA and RNA to probe their interactions with proteins”, Current Opinion in Structural Biology, 13:266, (2003); which is incorporated herein by reference). In a preferred embodiment, the straightening is performed using the fluid flow method.

### Example 4

### Formation of Multiple Individual Rows of a 2D Array

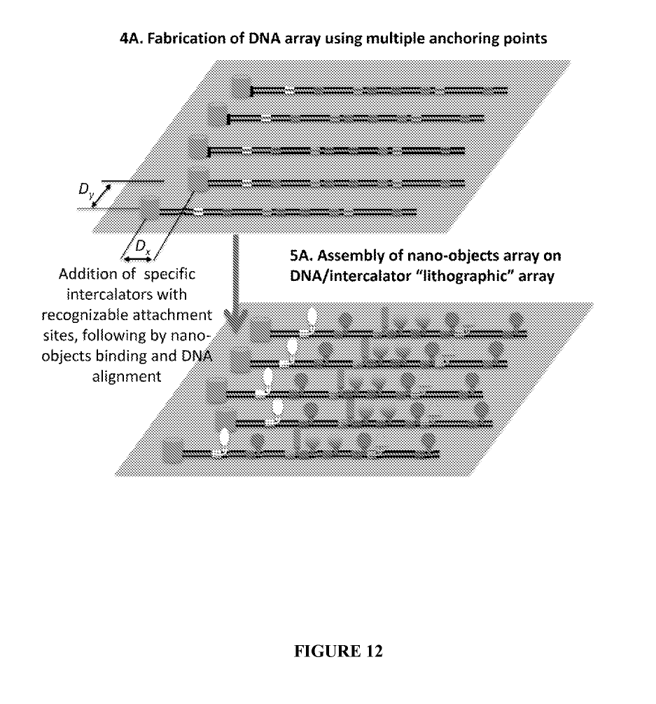

Multiple rows of a 2D array can be fabricated in the manner shown in FIG. 12 using a lithographic DNA. The steps described can be performed in any order. In a preferred embodiment, the steps are performed as set forth below.

Using multiple anchoring points and following a similar approach as described above, 2D arrays can be also fabricated (FIG. 12, top). Regular, periodic and non-periodic 1D patterns can be fabricated using anchoring points aligned in one line with designed separation (Dy) or shifted relative each other (Dx) (FIG. 12, top). The placement of anchoring points using traditional lithographic methods can be performed with tens of nm precision routinely. This is typically done using e-beam writer that burn (“write”) a defined area in a resist polymer layer in the pre-determined positions. In the next step material (typically metal, like gold) is deposited on a surface, and then a polymer layer is removed. This leaves the metal (gold) deposited spot, which is used as anchoring point.

This technique allows for programmable fabrication of large DNA encoded arrays containing a large number (up to about a million or more) of various nano-objects using only very simple fabrication of a relatively small number (about one hundred to about one thousand) of identical anchoring sites. These arrays can be on the scale of tens of microns in size. The size of arrays is determined by the choice of DNA. It can range from tens on nanometers to hundred of microns, or may be even larger. The preferable scale from characterization perspective and integration with other technologies is few microns and more. The upper limit is probably determined only by computation power required to choose suitable attachment sites and by easy available DNA. This approach is highly suitable for deposition of similar lithographic DNA if the same binding motif is used for attachment to anchoring points. Additionally, the specificity of interactions between the anchoring point and a DNA end can be designed thereby allowing multiple types of lithographic DNA to be used. For example, this can be accomplished by using DNA and proteins which allows for fabrication of significantly more complex structures due to incorporation at various DNA “rows”.

In a subsequent step (FIG. 12, bottom), intercalators can be added which recognize intercalator binding sites on the row DNA. This can then be followed by nano-objects binding and DNA alignment as discussed previously.

An advantage of the method (in particular FIG. 12, top and bottom) is the absence of any kind of conventional nano-fabrication except for fabrication of a first anchoring point. This method permits a fabrication of arbitrary placement of a large number of multiple types of nano-objects on at least tens of micron array with nm level-precision.

The DNA can be aligned in order to minimize its large scale bends. The DNA straightening step can be performed at any stage. In a preferred embodiment, the DNA is straightened after all nano-objects are assembled on lithographic ds-DNA.

The DNA can be straightened using a fluid flow, an electric field, or by optical tweezers (ALLEMAND, J. F., et al, “Stretching DNA and RNA to probe their interactions with proteins”, Current Opinion in Structural Biology, 13:266, (2003); which is incorporated herein by reference). In a preferred embodiment, the straightening is performed using the fluid flow method.

### Example 5

### Formation of a 2D Matrix Comprising a Column and Rows

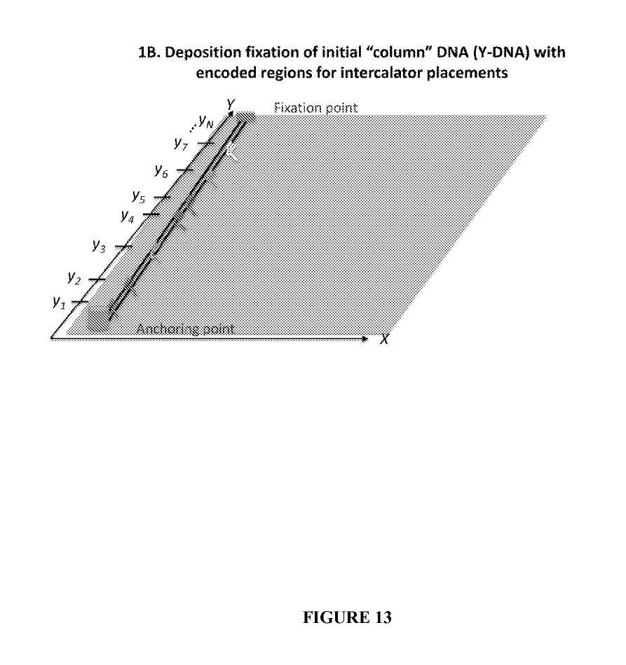

A 2D matrix comprising a column and rows can be fabricated in the manner shown in FIGS. 13-15 using a lithographic DNA. The steps described can be performed in any order. In a preferred embodiment, the steps are performed as set forth below.

Specifically, in an embodiment of the present invention, the need for fabrication multiple anchoring points (i.e., multiple individual rows of DNA attached by anchoring points) is eliminated. This design allows for a full scale 2D matrix formed by self-assembly. In this embodiment, the positioning of the individual DNA rows can be encoded by an appropriate choice of a column DNA (FIG. 13).

In the first step (FIG. 13), an initial lithographic ssDNA having a pre-designed sequence is vertically aligned with one end attached to the surface through an anchoring point and the other end attached through a fixation point. The fixation points can be chemically different from the anchoring points. Additionally, the termination sites on DNA that responsible for attachment to the points can also be chemically different. In some embodiments, it is not necessary to fix the second end since after straightening and drying the DNA is immobilized on the surface. In a specific embodiment, the full 2-side fixation is utilized when other in-liquid manipulations will be performed, for example, adding some perpendicular DNA lines. The initial lithographic ssDNA forms the first column of the matrix. Different regions of the column DNA encode positions (i.e., pre-designed sequences) where row DNA will later be attached using intercalators (e.g., PNA).

In some embodiments, the ends of row DNA are complementary (sticky) to the free tails intercalators that are attached to the column DNA. The row DNA will entropically recognize the correct positions on the column by binding to specific intercalators through Watson-Crick interactions due to the presence of the ssDNA end at the termination, which can be achieved either via intercalators with free ssDNA ends, or by biochemical cleavage DNA end. The column DNA can also contain intercalators with free ssDNA ends which are complimentary to those on row DNAs. Finally, intercalators and encoded nano-objects (e.g., nanoparticles) are introduced and find their programmed placed on row DNAs, whereby arbitrary matrix of nanoparticles is formed.

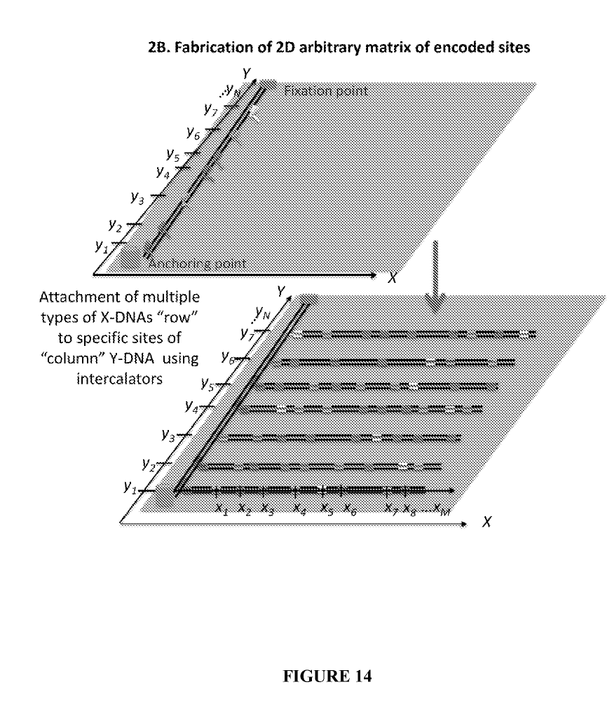

In a preferred embodiment, an initial lithographic DNA is attached to anchoring point with one end and a fixation point at the other end to form the column DNA (FIG. 13). The DNA can be either single stranded or double stranded. In a preferred embodiment the DNA is double stranded. In the next step (FIG. 14, top panel), an assembly of intercalators bind to the intercalator binding sites on the lithographic DNA at the pre-designed locations. Then, a set of various ds-DNAs, which contain either the same or different 1D positional encoding sites, is added (FIG. 14, bottom panel). One end of each of these row DNAs indirectly attaches to the column DNA by directly binding to the nano-object binding site on the intercalator, for example via PNA invasion. This results in assembly of 2D arbitrary matrix of DNA encoded binding sites. The position and binding specificity of the row DNA are also determined by design, as described previously. The accuracy of placement of row DNAs is similar to or less than the accuracy of nano-objects, as described previously.

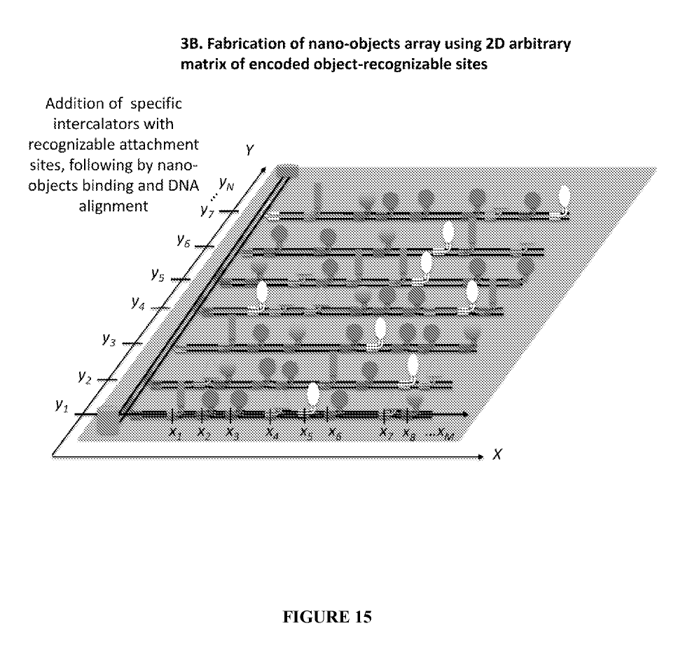

In a subsequent step (FIG. 15), additional intercalators can be added to the 2D array which recognize intercalator binding sites on the row DNA. The addition of specific intercalators, as discussed before in FIGS. 10 and 12, will allow for precise placement of multiple types of nano-objects on the row DNA. A simultaneous alignment of all row DNA with attached nano-objects can be performed at the final stage using known methods, as discussed above (e.g., FIG. 11). This will result in the formation of arbitrary arrays with various nano-objects on a fully designed architecture.

The DNA can be aligned in order to minimize its large scale bends. The DNA straightening step can be performed at any stage. In a preferred embodiment, the DNA is straightened after all nano-objects are assembled on lithographic ds-DNA.

The DNA can be straightened using a fluid flow, an electric field, or by optical tweezers (ALLEMAND, J. F., et al, “Stretching DNA and RNA to probe their interactions with proteins”, Current Opinion in Structural Biology, 13:266, (2003); which is incorporated herein by reference). In a preferred embodiment, the straightening is performed using the fluid flow method.

It will be appreciated by persons skilled in the art that the present description is not limited to what has been particularly shown and described in this specification. Rather, the scope is defined by the claims which follow. It should further be understood that the above description is only representative of illustrative examples of embodiments. For the reader's convenience, the above description has focused on a representative sample of possible embodiments, a sample that teaches the principles of the present invention. Other embodiments may result from a different combination of portions of different embodiments. The description has not attempted to exhaustively enumerate all possible variations. That alternate embodiments may not have been presented for a specific portion of the invention, and may result from a different combination of described portions, or that other undescribed alternate embodiments may be available for a portion, is not to be considered a disclaimer of those alternate embodiments. It will be appreciated that many of those undescribed embodiments are within the literal scope of the following claims, and others are equivalent. Furthermore, all references, publications, U.S. patents, and U.S. patent Publications cited throughout this specification are incorporated by reference in their entireties as if fully set forth in this specification.

1. An array comprising:
a surface having an anchoring point;
a strand of nucleic acids attached to the surface at the anchoring point;
an intercalator; and
a nano-object,
wherein one end of the intercalator binds to a specific sequence on the strand of nucleic acids and a second end of the intercalator binds to the nano-object.

2. The array according to claim 1, wherein the surface is a solid support made of silicon.

3. The array according to claim 1, wherein the anchoring point is a nucleic acid sequence, biotin, or streptavidin.

4. The array according to claim 1, wherein the strand of nucleic acids is DNA.

5. The array according to claim 1, wherein the strand of nucleic acids is a lithographic DNA.

6. The array according to claim 1, wherein the intercalator is a strand of nucleic acids, a protein, an organic compound, or a combination thereof.

7. The array according to claim 1, wherein the intercalator is a PNA-DNA chimera.

8. The array according to claim 1, wherein the nano-object is a nanoparticle, nanohorn, nanotube, or nanosphere.

9. The array according to claim 1, wherein the nano-object is a DNA-functionalized gold nanoparticle.

10. The array according to claim 1, wherein the surface is made of silicon, the strand of nucleic acids is a lithographic DNA, the intercalator is a PNA-DNA chimera, and the nano-object is a DNA-functionalized nanoparticle.

11. A method for assembling nano-objects on the array comprising:
preparing an array that comprises a surface having an anchoring point;
binding a strand of nucleic acids to the anchoring point on the surface; and
attaching a nano-object to a specific sequence on the strand of nucleic acids through an intercalator;
wherein one end of the intercalator binds to a specific sequence on the strand of nucleic acids and a second end of the intercalator binds to the nano-object.

12. The method according to claim 11, wherein the surface is made of silicon, the strand of nucleic acids is a lithographic DNA, the intercalator is a PNA-DNA chimera, and the nano-object is a DNA-functionalized nanoparticle.

13. The method according to claim 11, wherein the anchoring point is DNA and the strand of nucleic acids is bound to the anchoring point through DNA-DNA hybridization.

14. The method according to claim 11, wherein the strand of nucleic acids is bound to the surface by biotin-streptavidin interaction, thiointeration, or nucleic acid hybridization.

15. The method according to claim 11, wherein more than one nano-object is bound to the strand of nucleic acids at periodic or non-periodic intervals.

16. The method according to claim 11, wherein the array is 1D or 2D.

17. The method according to claim 11, wherein the surface is a solid support made of silicon.

18. The method according to claim 11, wherein the anchoring point is a nucleic acid sequence, biotin, or streptavidin.

19. The method according to claim 11, wherein the intercalator is a strand of nucleic acids, a protein, an organic compound, or a combination thereof.

20. The method according to claim 11, wherein the nano-object is a nanoparticle, nanohorn, nanotube, or nanosphere.

21. The method according to claim 11, wherein the nano-object is a DNA-functionalized gold nanoparticle.


SEQUENCE LISTING (8 sequences)

SEQ ID NO: 1 - DNA, 50 bp, Artificial Sequence
  Oligonucleotide
attgttattagctccacgccttctacatctgacgtttttttttttttttt

SEQ ID NO: 2 - DNA, 27 bp, Artificial Sequence
  Oligonucleotide
tgtagaaggcgtggagctaataacaat

SEQ ID NO: 3 - DNA, 30 bp, Artificial Sequence
  Oligonucleotide
tttttttttttttttttcagaagagatgtg

SEQ ID NO: 4 - DNA, 200 bp, Artificial Sequence
  Oligonucleotide
tccgcaagctggccctcacttcaacgcattattgttaatcttccaatgggccacctaccgtagacacggactctctacgcgttatgcctcagcatattattgttactgcgggacatacgatagagctttgctaaaataagtccctgcctttccaccaatagaaattattgttacgtagccaatcgacgtatttggtacgt

SEQ ID NO: 5 - DNA, 200 bp, Artificial Sequence
  Oligonucleotide
acgtaccaaatacgtcgattggctacgtaacaataatttctattggtggaaaggcagggacttattttagcaaagctctatcgtatgtcccgcagtaacaataatatgctgaggcataacgcgtagagagtccgtgtctacggtaggtggcccattggaagattaacaataatgcgttgaagtgagggccagcttgcgga

SEQ ID NO: 6 - DNA, 10 bp, Artificial Sequence
  Oligonucleotide
taataacaat

SEQ ID NO: 7 - DNA, 30 bp, Artificial Sequence
  Oligonucleotide
tttttttttttttttcacatctcttctgaa

SEQ ID NO: 8 - DNA, 10 bp, Artificial Sequence
  Oligonucleotide
cacatctctt